(19) United States
(12) Patent Application Publication
(10) Pub. No.: US 2008/0118442 A1
(43) Pub. Date: May 22, 2008

(54) Aerosol Formulations for Delivery of Dihydroergotamine to the Systemic Circulations Via Pulmonary Inhalation

(75) Inventors: Nahed Mohsen, Plymouth, MI (US); Thomas A. Armer, Cupertino, CA (US); Richard M. Pavkov, Therwil (CH)

Correspondence Address: MORRISON & FOERSTER LLP, 755 PAGE MILL RD, PALO ALTO, CA, 94304-1018, US

(73) Assignee: MAP PHARMACEUTICALS, INC., Mountain View, CA (US)

(21) Appl. No.: 10/572,012

(22) PCT Filed: Sep. 10, 2004
(86) PCT No.: PCT/US04/29632

§ 371 Date: Oct. 10, 2007

Related U.S. Application Data
(60) Provisional application No. 60/501,938, filed on Sep. 10, 2003.

(51) Int. Cl. A61K 9/12; A61P 25/06

(52) U.S. Cl. 424/46

(57) ABSTRACT

Pharmaceutical aerosol formulations of dihydroergotamine, or pharmaceutically acceptable salts thereof, to administer dry powders and propellant suspensions via pulmonary aerosol or nasal spray inhalation. Such formulations may be used for the treatment of various disease states and conditions, including, but not limited to, migraine headaches. The dihydroergotamine particles are produced using a supercritical fluid process. The aerosol formulations disclosed have superior stability, purity and comprise particle of respirable size particularly suitable for pulmonary delivery.

This application claims priority to and the benefit of U.S. provisional patent application No. 60/501,938, filed on Sep. 10, 2003.

### FIELD OF THE DISCLOSURE

The present disclosure relates to pharmaceutical aerosol formulations of dihydroergotamine, or pharmaceutically acceptable salts thereof, for pulmonary inhalation administration.

### BACKGROUND

The administration of serotonin agonists is well established for the treatment a variety of disease states and conditions, including, but not limited to, the treatment of acute migraine headache. The serotonin agonists most widely used are the triptans, including sumatriptan, zolmitriptan, naratriptan, rizatriptan, eletriptan, frovatriptan and almotriptan. These compounds bind specifically to serotonin 5-HT_{1D/1B} receptors. To a lesser degree, ergot alkaloids such as ergotamine tartrate and dihydroergotamine are also used for a variety of disease states and conditions, including, but not limited to the treatment of acute migraine. Dihydroergotamine is used extensively to treat chronic daily headache, formerly referred to as “transformed” migraine. The ergot alkaloids are less selective than the triptans with binding to 5-HT_{1D}, 5-HT_{1A}, 5-HT_{2A}, 5-HT_{2C}, noradrenaline α_{2A}, α_{2B}, and α, dopamine D_{2L} and D₃ receptors.

The ergot alkaloids have been less used, despite their potential benefit, in part because of the difficulty in stabilizing these compounds in a suitable formulation for delivery. Problems in stabilization result in inconsistent delivery and inconsistent dosing of the ergot alkaloid compounds. Dihydroergotamine has been used with oral and intranasal administration (Migranal®—Novartis, U.S. Pat. No. 5,942,251, EP0865789A3, and BE1006872A), but it is most often administered by intramuscular injection or by intravenous administration (D.H.E. 45®-Novartis). Recently, formulations of dihydroergotamine by itself and in combination with nonsteroidal analgesics have been developed for intramuscular autoinjectors (US Application 20030040537, U.S. Pat. No. 6,077,539, WO005781A3, EP1165044A2, CN1347313T, and AU0038825A5). Dihydroergotamine by itself or in combination with potent analgesics had also been formulated for treatment by intranasal administration (U.S. Pat. No. 4,462,983, U.S. Pat. No. 5,756,483, EP0689438A1, AU6428894A1, and WO9422445A3). Spray or aerosol formulations have also been developed for the sublingual administration of dihydroergotamine (US Application 20030017994). Ergotamine tartrate has been administered by injection, rectally with suppositories and via inhalation with metered dose inhaler (Medihaler-Ergotamine®-3M), but is most commonly administered orally or sublingually.

Ergotamine and dihydroergotamine have very low rectal, oral, sublingual and intranasal bioavailability—only 2% to 10% of the administered dose reaches the systemic circulation. Because injections are painful, cause local inflammation, reduce compliance, and because administration by IV requires costly clinical supervision, it would be very desirable to administer the ergot alkaloids by pulmonary inhalation. Pulmonary inhalation of the ergot alkaloids would minimize 1^{st} pass metabolism before their drugs can reach the target receptors because there is rapid transport from the alveolar epithelium into the capillary circulation and because of the relative absence of mechanisms for metabolism of the ergot alkaloid compounds in the lungs. Pulmonary delivery has been demonstrated to result in up to 92% bioavailability in the case of ergotamine tartrate. Pulmonary inhalation administration would also avoid gastrointestinal intolerance typical of migraine medications and minimize the undesirable taste experienced with nasal and sublingual administration due to the bitterness of the ergot alkaloid compounds. Pulmonary inhalation would minimize the reluctance to administer treatment associated with the invasiveness of injection and the cost of clinical supervision.

There are numerous recent citations of ergotamine tartrate formulations for administration via inhalation (U.S. Pat. No. 6,461,59, U.S. Pat. No. 6,451,287, U.S. Pat. No. 6,395,300, U.S. Pat. No. 6,395,299, U.S. Pat. No. 6,390,291, U.S. Pat. No. 6,315,122, U.S. Pat. No. 6,179,118, U.S. Pat. No. 6,119,853, U.S. Pat. No. 6,406,681) and specifically in propellant based metered dose inhaler (MDI) formulations (U.S. Pat. No. 5,720,940, U.S. Pat. No. 5,683,677, U.S. Pat. No. 5,776,434, U.S. Pat. No. 5,776,573, U.S. Pat. No. 6,153,173, U.S. Pat. No. 6,309,624, U.S. Pat. No. 6,013,245, U.S. Pat. No. 6,200,549, U.S. Pat. No. 6,221,339, U.S. Pat. No. 6,236,747, U.S. Pat. No. 6,251,368, U.S. Pat. No. 6,306,369, U.S. Pat. No. 6,253,762, U.S. Pat. No. 6,149,892, U.S. Pat. No. 6,284,287, U.S. Pat. No. 5,744,123, U.S. Pat. No. 5,916,540, U.S. Pat. No. 5,955,439, U.S. Pat. No. 5,992,306, U.S. Pat. No. 5,849,265, U.S. Pat. No. 5,833,950, U.S. Pat. No. 5,817,293, U.S. Pat. No. 6,143,277, U.S. Pat. No. 6,131,566, U.S. Pat. No. 5,736,124, U.S. Pat. No. 5,696,744). Many of these references require excipients or solvents in order to prepare stable formulations of the ergotamine tartrate. In the late 1980s 3M developed, received approval for and marketed a pulmonary inhalation formulation of an ergotamine tartrate (Medihaler-Ergotamine®-3M). It was removed from the market in the 1990s due to difficulties with inconsistent formulation and the resulting inconsistent dosing issues inherent therein.

Powders for inhalation in dry powder inhalation devices using ergotamine tartrate have also been described (U.S. Pat. No. 6,200,293, U.S. Pat. No. 6,120,613, U.S. Pat. No. 6,183,782, U.S. Pat. No. 6,129,905, U.S. Pat. No. 6,309,623, U.S. Pat. No. 5,619,984, U.S. Pat. No. 4,524,769, U.S. Pat. No. 5,740,793, U.S. Pat. No. 5,875,766, U.S. Pat. No. 6,098,619, U.S. Pat. No. 6,012,454, U.S. Pat. No. 5,972,388, U.S. Pat. No. 5,922,306). An aqueous aerosol ergotamine tartrate formulation for pulmonary administration has also been described (U.S. Pat. No. 5,813,597).

Despite these numerous references to aerosol delivery of ergotamine tartrate for pulmonary inhalation, there are few descriptions of delivery of dihydroergotamine via pulmonary inhalation (U.S. Pat. No. 4,462,983). While it would seem obvious to deliver dihydroergotamine in the same manner as ergotamine tartrate, dihydroergotamine has been very difficult to stabilize in the available aerosol delivery dosage forms. To maintain potency and activity the dihydroergotamine must be formulated in a solution, powder or suspension that can be stabilized without excipients or with excipients that do not affect the potency of dihydroergotomine and that are not toxic to the lungs. Dihydroergotamine is extremely sensitive to degradation and will degrade on exposure to light, oxygen and heat, or on exposure to oxidative or hydrolytic conditions. Aqueous formulations for delivery of dihydroergotamine by nasal sprays or by injection require chelating or complexing agents, such as caffeine, dextran or cyclodextrans, to stabilize the dihydroergotamine in solution. Such stabilization agents are often incompatible with pulmonary delivery because such stabilization agents cause local inflammation or are acutely toxic. To further inhibit the degradation of dihydroergotamine solutions, the dihydroergotomine formulations are sealed in dark-glass vials that must be opened with a specialized opener, filtered to remove glass shards, and transferred to injector or spray applicator just before use. Alternatively, the dihydroergotamine solution can be prepared just prior to use by mixing dihydroergotamine powder with injection fluid such as in a biphasic autoinjector format (powder portion is mixed with the liquid within a glass vial, syringe or blister package (such as the Pozen MT300). Such extemporaneous formulation approaches could be attempted to generate a solution for pulmonary delivery by jet or ultrasonic nebulization. However, any of the known nebulization processes used to generate inhalation aerosols from aqueous solutions expose the dihydroergotamine to sufficient heat and oxygen concentrations to cause immediate, variable changes in potency and activity. Because of these intrinsic difficulties in obtaining or aerosolizing a stable formulation, dihydroergotamine has not been suitable for administration via pulmonary inhalation.

Another method of aerosol deliver uses the pressurized metered dose inhaler (pMDI) wherein a halocarbon propellant forces a solution or suspension of the drug through a small orifice generating a fine inhalable mist consisting of the drug within the propellant droplets. To make stable pMDI formulations, the drug must be able to form solutions or fine particle suspensions that are stable in and physicochemically compatible with the propellant and the pMDI valve apparatus. Solution stability and lung toxicity issues described above for nasal or injection solutions are equally applicable to pMDI formulations, and the added requirement of propellant compatibility prohibits the use of accepted lung compatible reagents such as water or alcohol. For suspensions, fine particles of less than approximately 5.8 microns (mass median aerodynamic diameter necessary for deep lung penetration) are required, and the particle must be stable in the suspension. Such particles are generated from the bulk drug by attrition processes such as grinding, micronizing, milling, or by multiphase precipitation processes such as spray drying, solution precipitation, or lyophilization to yield powders that can be dispersed in the propellant. These processes often directly alter the physicochemical properties of the drug through thermal or chemical interactions. As dihydroergotamine is a very unstable compound, these process have not proven suitable for generating powders that can be redispersed in the propellant, or if the powder is initially dispersible, the particles grow in size over time, or change their chemical composition on exposure to the formulation over time. This instability caused changes in potency, activity, or increases the particle size above 3.0 microns making pMDI suspension formulation approaches unsuitable for dihydroergotamine aerosol delivery.

An additional method to generate respirable aerosols is to use dry powder inhalers wherein a powdered formulation of the drug is dispersed in the breath of the user and inhaled into the lungs. The difficulties described above for pMDI suspension formulations are equally applicable to generating stable dry powder formulation.

Clearly, the art is lacking a suitable formulation for inhalation delivery of dihydroergotamine. The present disclosure describes novel, stable formulations of dihydroergotamine, or pharmaceutically acceptable salts thereof, to administer dry powders and propellant suspensions via pulmonary aerosol or nasal spray inhalation. Such formulations may be used for the treatment of various disease states and conditions, including, but not limited to, migraine headaches. In addition, methods of producing the novel formulations of dihydroergotamine, or pharmaceutically acceptable salts thereof, are also described.

### DETAILED DESCRIPTION

Active compounds which are administered by inhalation must penetrate deep into the lungs in order to show topical, or alternatively, systemic action. In order to achieve this, the particles of the active compound must have a diameter which does not exceed approximately 0.5-5.8 μm mass mean aerodynamic diameter (MMAD). Particles of this optimal size range are rarely produced during the crystallization step, and secondary processes are required to generate particles in the 0.5-5.8 μm range. Such secondary processes include, but are not limited to, attrition by jet milling, micronization and mechanical grinding, multiphase precipitation such as solution precipitation, spray drying, freeze-drying or lyophilization. Such secondary processes involve large thermal and mechanical gradients which can directly degrade the potency and activity of active compound, or cause topological imperfections or chemical instabilities that change the size, shape or chemical composition of the particles on further processing or storage. These secondary processes also impart a substantial amount of free energy to the particles, which is generally stored at the surface of the particles. This free energy stored by the particles produces a cohesive force that causes the particles to agglomerate to reduce this stored free energy. Agglomeration processes can be so extensive that respirable, active compound particles are no longer present in the particulate formulation or can no longer be generated from the particulate formulation due to the high strength of the cohesive interaction. This process is exacerbated in the case of inhalation delivery since the particles must be stored in a form suitable for delivery by an inhalation device. Since the particles are stored for relatively long periods of time, the agglomeration process may increase during storage. The agglomeration of the particles interferes with the re-dispersion of the particles by the inhaler device such that the respirable particles required for pulmonary delivery and nasal delivery cannot be generated.

Additionally, most of the pharmaceutically customary methods used to overcome the agglomeration effect, such as the use of carriers and/or excipients, cannot be used in pharmaceutical forms for inhalation, as the pulmonary toxicological profile of these substances is undesirable.

The present disclosure describes novel, stable formulations of dihydroergotamine, or pharmaceutically acceptable salts thereof, (referred to herein as DHE) to administer dry powders and propellant suspensions via pulmonary aerosol inhalation or nasal spray inhalation. In one embodiment, DHE is used as the mesylate salt. The DHE powder is generated using a supercritical fluid processes. Supercritical fluid processes offer significant advantages in the production of DHE particles for inhalation delivery. Importantly, supercritical fluid processes produce respirable particles of the desired size in a single step, eliminating the need for secondary processes to reduce particle size. Therefore, the respirable particle produced using supercritical fluid processes have reduced surface free energy, which results in a decreased cohesive forces and reduced agglomeration. The particles produced also exhibit uniform size distribution. In addition, the particles produced have smooth surfaces and reproducible crystal structures which also tend to reduce agglomeration.

Such supercritical fluid processes may include rapid expansion (RES), solution enhanced diffusion (SEDS), gas-anti solvent (GAS), supercritical antisolvent (SAS), precipitation from gas-saturated solution (PGSS), precipitation with compressed antisolvent (PCA), aerosol solvent extraction system (ASES), or any combinations of the foregoing. The technology underlying each of these supercritical fluid processes is well known in the art and will not be repeated in this disclosure. In one specific embodiment, the supercritical fluid process used is the SEDS method as described by Palakodaty et al. in US Application 2003 0109421.

The supercritical fluid processes produce dry particulates which can be used directly by premetering into a dry powder inhaler (DPI) format, or the particulates may be suspended/dispersed directly into a suspending media, such as a pharmaceutically acceptable propellant, in a metered dose inhaler (MDI) format. The particles produced may be crystalline or may be amorphous depending on the supercritical fluid process used and the conditions employed (for example, the SEDS method is capable of producing amorphous particles). As discussed above, the particles produced have superior properties as compared to particles produced by traditional methods, including but not limited to, smooth, uniform surfaces, low energy, uniform particle size distribution and high purity. These characteristics enhance physicochemical stability of the particles and facilitate dispersion of the particles, when used in either DPI format or the MDI format.

The particle size should be such as to permit inhalation of the DHE particles into the lungs on administration of the aerosol particles. In one embodiment, the particle size distribution is less than 20 microns. In an alternate embodiment, the particle size distribution ranges from about 0.050 microns to 10.000 microns MMAD as measured by cascade impactors; in yet another alternate embodiment, the particle size distribution ranges from about and preferably between 0.400 and 3.000 microns MMAD as measured by cascade impactors. The supercritical fluid processes discussed above produce particle sizes in the lower end of these ranges.

In the DPI format the DHE particles can be electrostatically, cryometrically, or traditionally metered into dosage forms as is known in the art. The DHE particle may be used alone (neat) or with one or more pharmaceutically acceptable excipients, such as carriers or dispersion powders including, but not limited to, lactose, mannose, maltose, etc., or surfactant coatings. In one preferred formulation, the DHE particles are used without additional excipients. One convenient dosage form commonly used in the art is the foil blister packs. In this embodiment, the DHE particles are metered into foil blister packs without additional excipients for use with a DPI. Typical doses metered can range from about 0.050 milligrams to 2.000 milligrams, or from about 0.250 milligrams to 0.500 milligrams. The blister packs are burst open and can be dispersed in the inhalation air by electrostatic, aerodynamic, or mechanical forces, or any combination thereof, as is known in the art. In one embodiment, more than 25% of the premetered dose will be delivered to the lungs upon inhalation; in an alternate embodiment, more 50% of the premetered dose will be delivered to the lungs upon inhalation; in yet another alternate embodiment, more than 80% of the premetered dose will be delivered to the lungs upon inhalation. The respirable fractions of DHE particles (as determined in accordance with the United States Pharmacopoeia, chapter 601) resulting from delivery in the DPI format range from 25% to 90%, with residual particles in the blister pack ranging from 5% or the premetered dose to 55% of the premetered dose.

In the MDI format the particles can be suspended/dispersed directly into a suspending media, such as a pharmaceutically acceptable propellant. In one particular embodiment, the suspending media is the propellant. It is desirable that the propellant not serve as a solvent to the DHE particles. Suitable propellants include C₁₋₄ hydrofluoroalkane, such as, but not limited to 1,1,1,2-tetrafluoroethane (HFA 134a) and 1,1,1,2,3,3,3-heptafluoro-n-propane (HFA 227) either alone or in any combination. Carbon dioxide and alkanes, such as pentane, isopentane, butane, isobutane, propane and ethane, can also be used as propellants or blended with the C₁₋₄ hydrofluoroalkane propellants discussed above. In the case of blends, the propellant may contain from 0-25% of such carbon dioxide and 0-50% alkanes. In one embodiment, the DHE particulate dispersion is achieved without surfactants. In an alternate embodiment, the DHE particulate dispersion may contain surfactants if desired, with the surfactants present in mass ratios to the DHE ranging from 0.001 to 10. Typical surfactants include the oleates, stearates, myristates, alkylethers, alkylarylethers, sorbates and other surfactants used by those skilled in the art of formulating compounds for delivery by inhalation, or any combination of the foregoing. Specific surfactants include, but are not limited to, sorbitan monooleate (SPAN-80) and isopropyl myristate. The DHE particulate dispersion may also contain polar solvents in small amounts to aid in the solubilization of the surfactants, when used. Suitable polar compounds include C₂₋₆ alcohols and polyols, such as ethanol, isopropanol, polypropylene glycol and any combination of the foregoing. The polar compounds may be added at mass ratios to the propellant ranging from 0.0001% to 4%. Quantities of polar solvents in excess of 4% may react with the DHE or solubilize the DHE. In one particular embodiment, the polar compound is ethanol used at a mass ratio to the propellant from 0.0001 to 1%. No additional water or hydroxyl containing compounds are added to the DHE particle formulations other than is in equilibrium with pharmaceutically acceptable propellants and surfactants. The propellants and surfactants (if used) may be exposed to water of hydroxyl containing compounds prior to their use so that the water and hydroxyl containing compounds are at their equilibrium points.

Standard metering valves (such as from Neotechnics, Valois, or Bespak) and canisters (such as from PressPart or Gemi) can be utilized as is appropriate for the propellant/surfactant composition. Canister fill volumes from 2.0 milliliters to 17 milliliters may be utilized to achieve dose counts from one (1) to several hundred actuations. A dose counter with lockout mechanism can optionally be provided to limit the specific dose count irrespective of the fill volume. The total mass of DHE in the propellant suspension will typically be in the range of 0.100 milligram to 2.000 milligram of DHE per 100 microliters of propellant. Using standard MDI metering valves ranging from 50 to 100 microliters dosing will result in metered doses ranging from 0.050 micrograms to 1.000 microgram per actuation. An actuator with breath actuation can preferably be used to maximize inhalation coordination, but it is not mandatory to achieve therapeutic efficacy. The respirable fraction of such MDIs would range from 25% to 75% of the metered dose (as determined in accordance with the United States Pharmacopoeia, chapter 601).

### EXAMPLES

The following examples illustrate certain embodiments of the disclosure and are not intended to be construed in a limiting manner.

### Example 1

### Stability of Dry Powder DHE

DHE particle were produced by the SEDS super critical fluid process as described by Palakadoty et al. (US Application 20030109421). The DHE particulate powder produced was assayed by HPLC to determine purity and the mass mean aerodynamic diameter was determined using an Aerosizer instrument under standard operating conditions known in the art. As can be seen in Table 1, on production, the DHE particles had a HPLC purity of 98.3% and a particle size of 1.131 microns (MMAD). The DHE particulate powder was subject to standard accelerated aging conditions of: (i) 3 months at 40 degrees Celsius and 75% relative humidity; and (ii) 25 degrees Celsius and 60% relative humidity. The DHE particles were placed in a tightly sealed dark glass container and placed in the appropriate incubation ovens for the 3 month period. At the end of the three month period, purity and particle size were again assessed as discussed above. As can be seen in Table 1, the sample incubated for 3 months at 40 degrees Celsius and 75% relative humidity had a purity of 102.0% and a particle size of 1.091 microns (MMAD). Likewise the sample incubated at 25 degrees Celsius and 60% relative humidity had a purity of 101.0% and a particle size of 1.044 microns (MMAD).

These data indicate the DHE particulate powder produced using the supercritical fluid technology had excellent redispersibility characteristics on initial production and after three months of accelerated environmental aging. Importantly, the DHE particles were stable and remained in the respirable size range for deep lung penetration (<3.0 microns) even after the three month accelerated environmental aging. Such results were quite surprising given the difficulty in producing suitable DHE particles by conventional means. These results indicate that DHE particulate powders produced using supercritical fluid technology are suitable for pulmonary delivery by the DPI format. Significantly, the DHE particulate powder tested contained no excipients, a significant advance over the prior art formulations. The same lot (no. 3801087) of DHE particulate powder tested above was used in the formulation examples for the MDI format as described below.

TABLE 1
Powder Stability with Accelerated Environmental Aging

| | HPLC | Particle Size |
|---|---|---|
| | Assay | (microns by |
| | (%) | Aerosizer) |
| Initial | 98.3 | 1.131 |
| 3 Months @ 40 C./75% RH | 102.0 | 1.091 |
| 3 Months @ 25 C./60% RH | 101.0 | 1.044 |

### Example 2

### Formulations of DHE for Pulmonary Delivery by MPI

As described above, various formulations of the DHE particles can be prepared, either with or without excipients, although it is preferred to produce formulations without added excipients (other than the propellant). The DHE particles used in the formulation were obtained from the same lot described in Example 1.

Each formulation was packaged in a PressPart coated AI canister equipped with a Bespak BK357 valve and a Bespak 636 actuator; the total volume per actuation was 100 μl. The formulations exemplifying the teachings of the present disclosure are listed in Table 2, with performance characteristics of these formulations given in Table 3. The formulations listed in Table 2 should not be construed as limiting the present disclosure and the scope of the appended claims in any way and are given as examples of particular embodiments only to illustrate the teachings of the present disclosure. The DHE formulations were produced as described in the general methods set forth below. Both amorphous DHE particles and crystalline DHE particles were used in the formulations described in Table 2, as well micronized crystalline DHE particles produced by non supercritical fluid methods.

TABLE 2

| | Dihydroergatoamine | Isopropyl | | | | |
|---|---|---|---|---|---|---|
| | Mesylate* | Myristate | SPAN-80 | Ethanol | p134a | p227 |
| | (milligrams) | (milligrams) | (milligrams) | (milligrams) | (grams) | (grams) |
| 1 | 50.0 (SCF Amorphous) | 1.0 | 0.0 | 0.0 | 0.0 | 12.00 |
| 2 | 50.0 (SCF Crystalline) | 0.0 | 0.0 | 0.0 | 0.0 | 12.00 |
| 3 | 50.0 (SCF Crystalline) | 1.0 | 0.0 | 0.0 | 12.0 | 0.00 |
| 4 | 50.0 (SCF Amorphous) | 0.0 | 0.0 | 0.0 | 12.0 | 0.00 |
| 5 | 50.0 (Micronized | 0.2 | 0.0 | 0.0 | 12.0 | 0.00 |
| | Crystalline) | | | | | |
| 6 | 50.0 (Micronized | 0.0 | 0.0 | 0.0 | 12.0 | 0.00 |
| | Crystalline) | | | | | |
| 7 | 50.0 (SCF Crystalline) | 1.0 | 0.0 | 0.0 | 6.0 | 6.0 |
| 8 | 50.0 (SCF Amorphous) | 0.0 | 0.0 | 0.0 | 6.0 | 6.0 |
| 9 | 50.0 (SCF Crystalline) | 1.0 | 0.0 | 0.0 | 6.0 | 6.0 |
| 10 | 50.0 (SCF Crystalline) | 0.5 | 0.0 | 0.0 | 12.0 | 0.0 |
| 11 | 50.0 (SCF Crystalline) | 0.2 | 0.0 | 0.0 | 12.0 | 0.0 |
| 12 | 50.0 (SCF Crystalline) | 1.0 | 0.0 | 0.0 | 8.4 | 3.6 |
| 13 | 50.0 (SCF Crystalline) | 0.5 | 0.0 | 0.0 | 8.4 | 3.6 |
| 14 | 50.0 (SCF Crystalline) | 0.2 | 0.0 | 0.0 | 8.4 | 3.6 |
| 15 | 50.0 (SCF Crystalline) | 1.0 | 0.0 | 0.0 | 3.6 | 8.4 |
| 16 | 50.0 (SCF Crystalline) | 0.5 | 0.0 | 0.0 | 3.6 | 8.4 |
| 17 | 50.0 (SCF Crystalline) | 0.2 | 0.0 | 0.0 | 3.6 | 8.4 |
| 18 | 50.0 (SCF Crystalline) | 0.0 | 0.0 | 0.0 | 3.6 | 8.4 |
| 19 | 50.0 (SCF Crystalline) | 0.0 | 1.0 | 0.0 | 6.0 | 6.0 |
| 20 | 50.0 (SCF Crystalline) | 0.0 | 1.0 | 0.0 | 3.6 | 8.4 |
| 21 | 50.0 (SCF Crystalline) | 0.0 | 1.0 | 0.0 | 8.4 | 3.6 |
| 22 | 50.0 (SCF Crystalline) | 0.0 | 1.0 | 0.1 | 6.0 | 6.0 |
| 23 | 50.0 (SCF Crystalline) | 0.0 | 1.0 | 0.1 | 3.6 | 8.4 |
| 24 | 50.0 (SCF Crystalline) | 0.0 | 1.0 | 0.1 | 8.4 | 3.6 |

The formulations were tested to determine the fine particle fraction and to determine the mean mass aerodynamic diameter of the DHE particles contained in the various formulations. The fine particle fraction was determined according to the methods and standards set for the in the United States Pharmacopoeia, chapter 601, using an Anderson cascade impactor (at 28.3 LPM). In Table 3, the fine particle fraction indicates the percentage of DHE particles that impact the detector that have a diameter of 4.8 microns or less. This approximates the amount of drug that would be delivered to the lung of a subject for any given formulation. The fine particle dose is the actual amount of drug delivered during the actuation step. The MMAD was determined using an Aerosizer using protocols standard in the art. As can be seen in Table 3, the composition of the DHE formulation significantly impacted the performance characteristics of the formulation.

The DHE crystalline particles produced by the SEDS supercritical fluid method generally showed superior results to the DHE amorphous particles produced by the same technique. Both the SEDS produced crystalline and amorphous particles (samples 1, 4 and 8) showed significantly enhanced performance as compared to the standard micronized crystalline DHE particles (samples 5 and 6). For example, sample number 5 (micronized crystalline DHE dispersed in 100% HFA134a plus 0.2 milligrams isopropyl myristate) had a fine particle fraction of only 3.1% and had particles of 5.7 microns (MMAD) as compared to sample number 10 (SEDS produced crystalline DHE dispersed in 100% HFA134a plus 0.2 milligrams isopropyl myristate) which bad a fine particle fraction of 44.6% (a 14.4 fold increase) and particles of 2.2 microns (MMAD). This comparison illustrates the problems encountered in the prior art in formulating DHE particles for delivery by pulmonary inhalation, namely the difficulty in obtaining respirable DHE particles. Particularly preferred formulations are samples 2 and 18. Sample 2 is SEDS produced crystalline DHE dispersed in 100% HFA227, while sample 18 is SEDS produced crystalline DHE dispersed in 70% HFA227/30% HFA134a mixture. Sample 2 showed a fine particle fraction of 41.2% with particles having a MMAD of 2.3 microns while sample 18 had a fine particle fraction of 47.9% and particles with a MMAD of 1.9 microns. Each of these formulations exhibits superior qualities for pulmonary delivery of DHE.

TABLE 3

| | | | | Mass |
|---|---|---|---|---|
| | | | Fine | Median |
| | Dihydroergatoamine | Fine Particle | Particle | Aerodyamic |
| | Mesylate* | Dose | Fraction | Diameter |
| | (milligrams) | (milligrams) | (%) | (microns) |
| 1 | 50.0 (SCF Amorphous) | 203.6 | 33.9 | 3.8 |
| 2 | 50.0 (SCF Crystalline) | 209.4 | 41.2 | 2.3 |
| 3 | 50.0 (SCF Crystalline) | 98.4 | 19.5 | 3.7 |
| 4 | 50.0 (SCF Amorphous) | 124.5 | 30.0 | 4.1 |
| 5 | 50.0 (Micronized Crystalline) | 21.7 | 3.1 | 5.7 |
| 6 | 50.0 (Micronized Crystalline) | 3.6 | 0.8 | 5.3 |
| 7 | 50.0 (SCF Crystalline) | 68.5 | 23.6 | 4.3 |
| 8 | 50.0 (SCF Amorphous) | 68.5 | 22.3 | 4.5 |
| 9 | 50.0 (SCF Crystalline) | 267 | 46.0 | 2.1 |
| 10 | 50.0 (SCF Crystalline) | 258 | 44.6 | 2.2 |
| 11 | 50.0 (SCF Crystalline) | 279 | 45.9 | 2.1 |
| 12 | 50.0 (SCF Crystalline) | 224.4 | 39.2 | 2.3 |
| 13 | 50.0 (SCF Crystalline) | 261.3 | 43.9 | 2.0 |
| 14 | 50.0 (SCF Crystalline) | 261.4 | 46.2 | 2.1 |
| 15 | 50.0 (SCF Crystalline) | 272.7 | 44.2 | 2.1 |
| 16 | 50.0 (SCF Crystalline) | 272.3 | 46.4 | 1.9 |
| 17 | 50.0 (SCF Crystalline) | 344.8 | 51.8 | 1.8 |
| 18 | 50.0 (SCF Crystalline) | 263.4 | 47.9 | 1.9 |
| 19 | 50.0 (SCF Crystalline) | 209.0 | 48.1 | 1.8 |
| 20 | 50.0 (SCF Crystalline) | 218.3 | 47.4 | 1.9 |
| 21 | 50.0 (SCF Crystalline) | 206 | 46.0 | 1.9 |
| 22 | 50.0 (SCF Crystalline) | 211.5 | 43.2 | 2.1 |
| 23 | 50.0 (SCF Crystalline) | 162.1 | 31.7 | 3.7 |
| 24 | 50.0 (SCF Crystalline) | 153.2 | 33.2 | 3.8 |

### Example 3

### Pulmonary Delivery of DHE

Upon delivery by either DPI or MDI a large fraction of the metered dose of the DHE particles (in the DPI embodiment) or DHE particulate dispersion (in the MDI embodiment) would be delivered to the peripheral lung (beyond the subbrochioli) with lesser fractions delivered to the central lung or conductive airways, and only a minor fraction delivered to the oropharyngeal biospace. For example, the fine particle fraction data from Table 3 indicate the percentage of the fraction of DHE that would have been administered to the lungs for each of the above formulations. As can be seen from Table 3, with crystalline DHE produced using the supercritical fluid processes described, a fraction from 31.7% to 51.8% of the total DHE dose would have been delivered to the lungs. In particular, samples 2 and 18 show a delivery fraction of 41.2% and 47.9% without the addition of surfactants and other materials (i.e. propellant only). A significant amount of the DHE would be delivered to the alveolar biospace such that rapid and efficient absorption into capillary circulation could occur. In one embodiment, peak blood or plasma concentrations of the DHE could occur within 5 to 10 minutes to effect rapid therapeutic action. Such pharmacokinetic response would be comparable to intravenous administration and significantly more rapid than oral administration (for 30 minutes to 2 hours), sublingual (30 minutes to 2 hours), intranasal (15 minutes to 30 minutes) and intramuscular injection (15 minutes to 25 minutes).

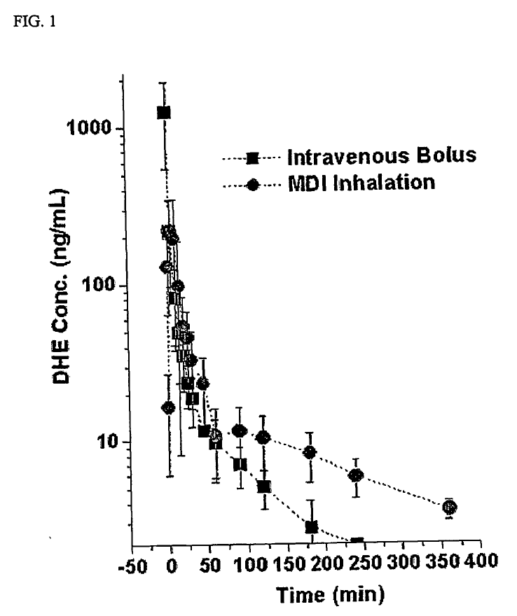

FIG. 1 shows pharmacokinetic data illustrating the rapid absorption of DHE particles delivered via dry powders. In this study, dogs were administered the DHE particles via the DPI format (total dose 1 mg) and by intravenous bolus (total dose 0.5 mg) and DHE levels were measured in dog serum at defined intervals. As can be seen in FIG. 1, measurable levels of DHE in the blood appear within 30 seconds after inhalation, with peak levels occurring 5 to 10 minutes after inhalation. Furthermore, the blood levels of DHE were maintained at higher levels over an extended period of time as compared to the intravenous delivery.

Table 4 below shows Tₘₐₓ and F (bioavailability) of DHE in dog serum after inhalation (n=3). As can be seen, Tₘₐₓ occurred at an average of 6.7 minutes (with a standard deviation of 2.9 minutes) and the bioavailability of the DIE was 52% (with a standard deviation of 27%). These results show superior pulmonary delivery and bioavailability of DHE via the inhalation route.

TABLE 4

| Tₘₐₓ | Average | SD | F* | Average | SD |
|---|---|---|---|---|---|
| (minutes) | (minutes) | (minutes) | (%) | (%) | (%) |
| 5 | 6.7 | 2.9 | 27 | 52 | 27 |
| 5 | | | 49 | | |
| 10 | | | 80 | | |

*F = (AUCᵢₕ/AUCⁱⁿ) * (Dᵢᵥ/Dᵢₕ), where “iv” corresponds to intravenous bolus and “ih” corresponds to inhalation. Dᵢᵥ = 0.5 mg; Dih = 1.0 mg; AUCᵢᵥ is the average AUC from 3 dogs.

### Preparation of Formulations

The following protocol outlines the manufacturing process for the formulations described in Tables 2 and 3. The following descriptions are provided by way of non-limiting example and are not meant to disclose other methodologies for preparing the formulations.

### HFA227

For formulations containing HFA227 as the propellant and with no added surfactants, the dry DHE powder is weighed into a mixing kettle (equipped with chilling jacket, Lightning Mixer, and a 3 port cover and situated on a weight scale). The kettle is chilled to 0 Celsius and blanketed with dry Nitrogen then filled with approximately 50% of the total mass of the HFA227 to be used. The HFA227 is pumped into the vessel under pressure of 500 millibars and at a temperature of approximately 0 Celsius through a stainless steel tube. The force of the HFA227 impacting the drug powder charge on the bottom of the kettle is sufficient to suspend/disperse the DHE powder into the propellant. When the HFA227 level in the kettle is sufficient to submerge the propeller of the lightning mixer, the mixer is energized to continuously stir the suspension at medium speed. After mixing for 20 minutes following the addition of the HFA227 (50% of the total volume to be used) the mixture is pumped into canisters to fill approximately 50% weight in each canister. The valves are crimped on the top of each canister and the balance of the p227 is filled under pressure through the stem of the valve to bring to 100% weight. The canisters are water tested, discharge tested, weigh checked and released for testing.

For formulations containing HFA227 plus surfactant, a mixing kettle (equipped with chilling jacket, a Silverstone Homogenizer, a Lightning Mixer, and a 4 port cover and situated on a weight scale) is chilled to 0 Celsius and blanketed with dry Nitrogen. The kettle is filled with HFA227 pumped in under pressure of 500 millibars and at a temperature of approximately 0 Celsius through a stainless steel tube until approximately 20% of the total mass of the HFA227 to be used is in the kettle. The surfactant is weighed separately and added to the HFA227 in the vessel under continuous stirring by the mixer. After complete addition of the surfactant the homogenizer is energized and the mixture is sonicated for approximately 20 minutes. Another 30% of the total p227 is pumped into the vessel under pressure of 500 millibars and at a temperature of approximately 0 Celsius through a stainless steel tube. The sonicator is deenergized and the lightning mixer is energized. The drug powder is added to the vessel and continuously stirred at medium speed. After mixing for 20 minutes the mixture is pumped into canisters to fill approximately 50% weight in each canister. The valves are crimped on the top of each canister and the balance of the p227 is filled under pressure through the stem of the valve to bring to 100% weight. The canisters are water tested, discharge tested, weigh checked and released for testing.

### HFA134a

For formulations containing HFA134a, the dry powder is weighed into a mixing kettle (equipped with chilling jacket, Lightning Mixer, and a 3 port cover and situated on a weight scale). The kettle is chilled to −27 Celsius, pressurized approximately 2000 millibars with dry Nitrogen then filled with approximately 50% of the total mass of the HFA134a to be used. The HFA134a is pumped into the vessel under pressure of 2500 millibars and at a temperature of approximately −27 Celsius through a stainless steel tube. The force of the HFA134a impacting the drug powder charge on the bottom of the kettle is sufficient to suspend/disperse the DHE particles in the propellant. When the HFA134a level in the kettle is sufficient to submerge the propeller of the lightning mixer the mixer is energized to continuously stir the suspension at medium speed. After mixing for 20 minutes following complete addition of 50% of the HFA134a, the mixture is pumped into canisters to fill approximately 50% weight in each canister. The valves are crimped on the top of each canister and the balance of the HFA134a is filled under pressure through the stem of the valve to bring to 100% weight. The canisters are water tested, discharge tested, weigh checked and released for testing.

For formulations containing HFA134a plus surfactant, a mixing kettle (equipped with chilling jacket, a Silverstone Homogenizer, a Lightning Mixer, and a 4 port cover and situated on a weight scale) is chilled to −27 Celsius and blanketed with dry Nitrogen. The kettle is filled with HFA134a pumped in under pressure of 2500 millibars and at a temperature of approximately −27 Celsius through a stainless steel tube until approximately 20% of the total mass of the HFA134a to be used is in the kettle. The surfactant is weighed separately and added to the HFA134a in the vessel under continuous stirring by the mixer. After complete addition of the surfactant the homogenizer is energized and the mixture is sonicated for approximately 20 minutes. Another 30% of the total HFA134a is pumped into the vessel under pressure of 2500 millibars and at a temperature of approximately −27 Celsius through a stainless steel tube. The sonicator is deenergized and the lightning mixer is energized. The drug powder is added to the vessel and continuously stirred at medium speed. After mixing for 20 minutes, the mixture is pumped into canisters to fill approximately 50% weight in each canister. The valves are crimped on the top of each canister and the balance of the HFA134a is filled under pressure through the stem of the valve to bring to 100% weight. The canisters are water tested, discharge tested, weigh checked and released for testing.

### HFA227 and HFA134a Mixtures

For formulations containing both HFA227 and HFA134a without surfactant, the dry powder is weighed into a mixing kettle (equipped with chilling jacket, Lightning Mixer, and a 3 port cover and situated on a weight scale). The kettle is chilled to 0 Celsius, pressurized approximately 500 millibars with dry Nitrogen then filled with approximately 100% of the total mass of the HFA227 to be used. The HFA227 is pumped into the vessel under pressure of 500 millibars and at a temperature of approximately 0 Celsius through a stainless steel tube. The force of the p227 impacting the drug powder charge on the bottom of the kettle is sufficient to suspend/disperse the DHE particles in the propellant. When the HFA227 level in the kettle is sufficient to submerge the propeller of the lightning mixer the mixer is energized to continuously stir the suspension at medium speed. After mixing for 20 minutes following complete addition of the HFA227, the mixture is pumped into canisters to fill approximately from 30% to 50%, to 70% of intended final weight in each canister (dependent upon the final weight ratio of the HFA134a/HFA227). The valves are crimped on the top of each canister and 100% of the mass of HFA134a is filled under pressure through the stem of the valve to bring to 100% weight. The canisters are sonicated for 15 minutes in an ultrasonic water bath, water tested, discharge tested, weigh checked and released for testing.

For formulations containing both HFA227 and HFA134a with surfactant, a mixing kettle (equipped with chilling jacket, a Silverstone Homogenizer, a Lightning Mixer, and a 3 port cover and situated on a weight scale) is chilled to 0 Celsius and blanketed with dry Nitrogen. The kettle is filled with HFA227 pumped in under pressure of 500 millibars and at a temperature of approximately 0 Celsius through a stainless steel tube until approximately 100% of the total mass of the HFA227 to be used is in the kettle. The surfactant is weighed separately and added to the HFA227 in the vessel under continuous stirring by the mixer. After complete addition of the surfactant the homogenizer is energized and the mixture is sonicated for approximately 20-40 minutes while cooling the kettle to −27 Celsius. Approximately 30% of the total HFA134a is pumped into the vessel under pressure of 2500 millibars and at a temperature of approximately −27 Celsius through a stainless steel tube. The sonicator is deenergized and the lightning mixer is energized. The drug powder is added to the vessel and continuously stirred at medium speed. After mixing for 20 minutes the mixture is pumped into canisters to fill approximately 50% weight in each canister. The valves are crimped on the top of each canister and the balance of the HFA134a is filled under pressure through the stem of the valve to bring to 100% weight. The canisters are water tested, discharge tested, weigh checked and released for testing.

### With Alcohol with or without Surfactant

For formulations containing polar compounds (such as alcohols), a mixing kettle (equipped with chilling jacket, a Silverstone Homogenizer, a Lightning Mixer, and a 3 port cover and situated on a weight scale) is chilled to 0 Celsius and blanketed with dry Nitrogen. The kettle is filled with HFA227 pumped in under pressure of 500 millibars and at a temperature of approximately 0 Celsius through a stainless steel tube until approximately 100% of the total mass of the HFA227 to be used is in the kettle. The surfactant and alcohol are weighed separately then mixed until the surfactant is dissolved. The surfactant/alcohol solution is pumped into the kettle using a precision metering pump over approximately 20 minutes under continuous stirring by the mixer. After complete addition of the surfactant/alcohol solution the homogenizer is energized and the mixture is sonicated for approximately 20-40 minutes while cooling the kettle to −27 Celsius. Approximately 30% of the total HFA134 is pumped into the vessel under pressure of 2500 millibars and at a temperature of approximately −27 Celsius through a stainless steel tube. The sonicator is deenergized and the lightning mixer is energized. The drug powder is added to the vessel and continuously stirred at medium speed. After mixing for 20 minutes the mixture is pumped into canisters to fill approximately 50% weight in each canister. The valves are crimped on the top of each canister and the balance of the HFA134 is filled under pressure through the stem of the valve to bring to 100% weight. The canisters are water tested, discharge tested, weigh checked and released for testing. In the special case of no surfactant the same procedures are followed except that no surfactant is added to the alcohol.

Given the disclosure herein, one of ordinary skill in the art may become aware of various other modifications, features, or improvements. Such other modifications, features and improvements should be considered part of this disclosure. The cited references are incorporated by reference as if fully disclosed herein.

What is claimed:

1-21. (canceled)

22. A pharmaceutical aerosol formulation for delivery by inhalation, said aerosol formulation comprising: (i) a particulate powdered medicament produced by a supercritical fluid process, said medicament being dihydroergotamine; and (ii) a hydrofluroalkane propellant, said particulate powdered medicament having a mean particle size of 10 microns or less.

23. The aerosol formulation of claim 22 where the dihydroergotamine is the mesylate salt.

24. The aerosol formulation of claim 22 where said supercritical fluid process is selected from the group consisting of: rapid expansion, solution enhanced diffusion, gas-anti solvent, supercritical antisolvent, precipitation from gas-saturated solution, precipitation with compressed antisolvent and aerosol solvent extraction system.

25. The aerosol formulation of claim 22 where said supercritical fluid process is solution enhanced diffusion.

26. The aerosol formulation of claim 22 where said hydrofluroalkane propellant is selected from the group consisting of: 1,1,1,2-tetrafluoroethane, 1,1,1,2,3,3,3-heptafluoro-n-propane and a mixture of 1,1,1,2-tetrafluoroethane and 1,1,1,2,3,3,3-heptafluoro-n-propane.

27. The aerosol formulation of claim 22 where said hydrofluoroalkane propellant is a mixture of 1,1,1,2-tetrafluoroethane and 1,1,1,2,3,3,3-heptafluoro-n-propane, said mixture containing 30% or less of 1,1,1,2-tetrafluoroethane.

28. The aerosol formulation of claim 22 where said hydrofluoroalkane propellant is a mixture of 1,1,1,2-tetrafluoroethane and 1,1,1,2,3,3,3-heptafluoro-n-propane, said mixture containing 30% or less of 1,1,2,3,3,3-heptafluoro-n-propane.

29. The aerosol formulation of claim 22 where said hydrofluroalkane propellant is 1,1,1,2-tetrafluoroethane.

30. The aerosol formulation of claim 22 where said hydrofluroalkane propellant is 1,1,1,2,3,3,3-heptafluoro-n-propane.

31. The aerosol formulation of claim 22 further comprising at least one compound having a higher polarity than said propellant.

32. The aerosol formulation of claim 22 further comprising ethanol.

33. The aerosol formulation of claim 32 where said ethanol is present at less than 1% (w/w) based on said propellant.

34. The aerosol formulation of claim 22 further comprising at least one excipient selected from the group consisting of: oleates, stearates, myristates, alkylethers, alkylarylethers, sorbates and mixtures thereof.

35. The aerosol formulation of claim 22 further comprising an excipient, said excipients being sorbitan monooleate

36. The aerosol formulation of claim 22 further comprising an excipient, said excipient being isopropyl myristate.

37. The aerosol formulation of claim 22 where said powdered particulate medicament exhibits a peak absorbance into the blood in less than 10 minutes.

38. The aerosol formulation of claim 22 where the powdered particulate medicament has a respirable fraction of 30% or more.

39. The aerosol formulation of claim 22 where the powdered particulate medicament has a respirable fraction of 50% or more.

40. The aerosol formulation of claim 22 administered by a metered dose inhaler.

41. The aerosol formulation of claim 22 which is free of surfactant.

42. The aerosol formulation of claim 22 where said particulate powdered medicament has a mean particle size of 3 microns or less.

43-52. (canceled)

53. A pharmaceutical dry-powder aerosol formulation for delivery by inhalation, said aerosol formulation comprising: (i) a particulate powdered medicament produced by a supercritical fluid process, said medicament being dihydroergotamine, said particulate powdered medicament having a mean particle size of less than 5 microns.

54. The dry-powder aerosol formulation of claim 53 where the dihydroergotamine is the mesylate salt.

55. The dry-powder aerosol formulation of claim 53 where said supercritical fluid process is selected from the group consisting of: rapid expansion, solution enhanced diffusion, gas-anti solvent, supercritical antisolvent, precipitation from gas-saturated solution, precipitation with compressed antisolvent and aerosol solvent extraction system.

56. The dry-powder aerosol formulation of claim 53 where said supercritical fluid process is solution enhanced diffusion.

57. The dry-powder aerosol formulation of claim 53 further including one or more pharmaceutically acceptable excipients.

58. The dry-powder aerosol formulation of claim 57 where said excipients are selected from the group consisting of: carriers and dispersion powders.

59. The dry-powder aerosol formulation of claim 57 where said excipients are selected from the group consisting of: lactose, mannose, maltose, and surfactant coatings.

60. The dry-powder aerosol formulation of claims 53 where said powdered particulate medicament exhibits a peak absorbance into the blood in less than 10 minutes.

61. The dry-powder aerosol formulation of claim 53 where the powdered particulate medicament has a respirable fraction of 30% or more.

62. The dry-powder aerosol formulation of claim 53 where the powdered particulate medicament has a respirable fraction of 50% or more.

63. (canceled)

64. A method for treating migraines, said method comprising administering a pharmaceutically acceptable amount of a pharmaceutical aerosol formulation as claimed in claim 22.

65. (canceled)

66. A method for treating migraines, said method comprising administering a pharmaceutically acceptable amount of a pharmaceutical dry powder aerosol formulation as claimed in claim 53.
